(19) United States
(12) Patent Application Publication
(10) Pub. No.: US 2011/0086802 A1
(43) Pub. Date: Apr. 14, 2011

(54) METHODS TO INCREASE PERMEABILITY OF CORNEAL EPITHELIUM AND DESTABILIZE STROMAL COLLAGEN FIBRIL NETWORK

(76) Inventors: Bruce Dewoolfson, Vienna, VA (US); Dale Devore, Chelmsford, MA (US)

(21) Appl. No.: 12/934,310

(22) PCT Filed: Mar. 18, 2009
(86) PCT No.: PCT/US2009/037497

§ 371 Date: Dec. 8, 2010

Related U.S. Application Data
(60) Provisional application No. 61/064,730, filed on Mar. 24, 2008.
(60) Provisional application No. 61/064,731, filed on Mar. 24, 2008.

(51) Int. Cl. A61K 38/39; A61K 31/365; A61K 31/194; A61P 27/02; A61K 38/17

(52) U.S. Cl. 514/17.2; 514/473; 514/470; 514/574; 514/20.8

(57) ABSTRACT

Methods of increasing the permeability of corneal epithelium to facilitate the diffusion of agents into the collagen fibrillar network of the stroma are provided. Used in combination, these methods open the epithelium to facilitate diffusion of stabilization molecules into the stroma and dissociate bridging molecules from stromal collagen fibers, thereby priming the collagen fibrillar network for restabilization by stabilization molecules. These methods can be used to increase the effectiveness and longevity of non-invasive corneal reshaping, such as orthokeratology, for correcting myopia, hyperopia and astigmatism.

This application claims priority to U.S. Provisional Application Nos. 61/064,730 filed Mar. 24, 2008, and 61/064,731 filed Mar. 24, 2008, the contents of which are all incorporated herein by reference.

### FIELD OF THE INVENTION

The present disclosure relates to methods of increasing the permeability of corneal epithelium to allow diffusion of molecules, such as collagen binding molecules, into the corneal stroma and to methods of temporarily destabilizing the collagen fibrillar network of the stroma. The treatments described herein (1) open the epithelium to enhance the diffusion of molecules into the stroma and (2) dissociate bridging molecules from stromal collagen fibers, thereby priming the collagen fibrillar network for restabilization by stabilization molecules. Such treatments are important to improve the effectiveness and longevity of non-invasive corneal reshaping to correct myopia, hyperopia and astigmatism.

### BACKGROUND

Orthokeratology procedure: Orthokeratology is a nonsurgical procedure to improve refractive errors of the eye, and is an alternative to, e.g., laser eye surgery. Specifically, orthokeratology is a therapeutic procedure to reshape the curvature of a patient's cornea. A conventional orthokeratology procedure involves the use of a series of progressive contact lenses that are intended to gradually reshape the cornea and produce a more spherical anterior curvature. The process typically involves the fitting of two to as many as several pairs of specially designed contact lenses, and it has traditionally taken approximately three to six months to achieve optical reshaping. This procedure has been proven to reduce or eliminate myopia and astigmatism, hence improving natural vision and producing emmetropia (a state where vision experiences zero refractive error, or where no correction is necessary). Recent improvements in orthokeratology lens designs make it possible to achieve emmetropia much more rapidly. In many cases, this may be accomplished with a single night's wear of a single pair of end result lenses.

A limitation of orthokeratology is that reshaped corneal tissue keeps a memory of its original curvature, and tends to relax and return to the original curvature after the lenses are removed. Therefore, when an orthokeratology patient reaches maximum results, retainer contact lenses are prescribed for part-time wear to stabilize the results. The retainer contact lenses have typically been made of rigid gas permeable material. Orthokeratology patients increasingly wear retainer contact lenses during the night to obtain the desired results quickly, and enjoy almost emmetropic vision during their daytime activities. A disadvantage of such a modality is that it requires the wearing of retainer lenses every night in order to keep the cornea from regressing to its former shape.

Corneoplasty: A related procedure directed to solve this problem uses a corneal softening agent to temporarily soften the cornea so that it can be more easily reshaped to a desired configuration to produce emmetropia. The corneoplasty procedure is a three-step process performed in one visit or over a period of several weeks. The three-step process includes: first, applying a softening agent to the cornea to soften corneal tissue; second, placing a rigid contact lens over the cornea to render the eye emmetropic; and third, applying a stabilizing agent. The cornea would then reshape and conform to the desired configuration dictated by the rigid contact lens. Administration of the corneal softening agent helps correct larger refractive errors in a shorter period of time.

However, it has been found that it is difficult to accurately place the shaping contact lens with respect to the axis of vision to control the reshaping of the corneal tissue. In some unsuccessful applications, corneoplasty has induced astigmatism or double vision due to errors caused by misplacing the shaping contact lens. In addition, because all three steps are performed in one visit, the patient lacks an opportunity to react to the result of reshaped corneal tissue. The patient cannot “try and see” or guide the clinician to help achieve a better outcome during the process.

In light of the foregoing, there is a need for an improved method for performing an acuity correcting procedure that enables the patient to quickly reach emmetropia, while retaining the option of reverting back to his prior level of vision, e.g., to make the procedure be reversible up until the patient chooses to have the correction made permanent. Furthermore, there is a need for compositions and methods for making and applying the composition that stabilizes the cornea matrix. The compositions need to be able to stabilize the corneal curvature resulting from the orthokeratology procedure so that an orthokeratology patient can dispense with wearing rigid retainer contact lenses, dispense with applying a softening agent, and yet retain the opportunity to regress to the original corneal curvature up until the patient is convinced that they want the correction made permanent.

U.S. Pat. Nos. 6,161,544 and 6,946,440 describe the application of molecules to stabilize the cornea following orthokeratology. In U.S. Pat. No. 6,946,440 (DeWoolfson and DeVore) the stabilization molecules are relatively high molecular weight, natural, extracellular matrix molecules that stabilize the stroma matrix following orthokeratology. Stabilization occurs due to ionic binding of these molecules between adjacent collagen fibrils, forming a crosslink (or bridge) between such fibers. However, the penetration of these extracellular matrix molecules is limited due to fact that the intrinsic conjunctional epithelial tissue layer forms tight junctions with high resistance to ocular delivery of hydrophilic molecules greater than 500 daltons. In addition, binding sites for the exogenous stabilization molecules on collagen fibers are limited since the sites are inherently occupied by natural extracellular matrix molecules.

Thus, in order to enhance delivery of stabilization agents and thereby maximize stromal stabilization, methods are needed to (1) open the epithelium to allow intrastromal penetration of the stabilization agents and (2) dissociate inherent binding of extracellular molecules between adjacent collagen fibers in stromal tissue. The methods of opening the epithelium are also beneficial in that they can be used to enhance the delivery of any ocular drug to the corneal stroma.

### Corneal Anatomy

The human cornea is composed of three primary layers; epithelium, stroma, and endothelium. The thickness of the cornea is normally 500-600 μm, 90% being stroma. The epithelium is approximately 50 μm thick and contains 5-6 layers of cells with tight junctures between the cells, especially the first 2 layers of flattened, plate-like superficial cells. The next 2-3 layers contain wing-like or polygonal cells over a single row of columnar basal cells.

The epithelium forms a permeability barrier, especially to polar and ionic molecules. For ionic and hydrophilic molecules, molecular size affects their ability to penetrate the epithelium. Permeability of such molecules is generally limited to a molecular size of about 500 daltons. (See Liaw and Robinson, In “Ophthalmic Drug Delivery Systems” Ed. A. K. Mitra, Marcel Dekker, Inc. NY, 1993) In contrast, lipophilic molecules are easily absorbed across the epithelium.

The next barrier below the epithelium is Bowman's Membrane. Bowman's Membrane is an 8-14 μm thick homogenous sheet separating the epithelium from the underlying, acellular stroma (substantia propria).

The stroma is composed of 200-250 alternating lamellae (layers) of collagen fibers. Each lamellae is about 1 μm thick and 10-25 μm wide. The stroma contains 70% water and impedes movement of molecules greater than about 500,000 daltons. Collagen fibers make up a majority of the structure of cornea. Proteoglycans and fiber associated collagens are linked to collagen fibers to control diameter and stabilize stromal architecture. Fiber associated proteoglycans include a category called small leucine-rich proteoglycans (SLRPs) and includes decorin, biglycan, keratocan, lumican, mimican, and fibromodulin. Fiber associated collagens encompass a category known as fibril associated collagen molecules with interrupted triple helices (FACITs) and includes Type VI, Type X, Type XII, and Type XIV collagen.

Corneal integrity can be compromised by sufficiently high concentrations of certain excipients including preservatives (benzalkonium chloride), cationic surfactants, and chelating agents (0.5% EDTA).

Godbey (p 102) disrupted the top layers of epithelial cells using 0.02% cetylpyridium chloride. Shih and Lee (p 86) stripped off layers of epithelium by pretreating cornea with digitonin to exfoliate the top 2 layers of epithelium. This treatment was found to enhance penetration of timolol.

Other penetration enhancers have included Cytochalasin B, a cytoskeletal modulator.

Delivery of hydrophilic molecules with poor permeability currently depends on noncorneal penetration. The noncorneal route of absorption involves penetration across the sclera and conjunctiva into intraocular tissues. This is an inefficient method of delivering agents to the cornea because when the agent penetrates the surface of the eye beyond the corneal-scleral limbus, it is picked up by local capillary beds and removed by general circulation. Generally, less than 1% of ophthalmic solutions depending on noncorneal absorption reach the aqueous humor. For a review of some of the difficulties associated with the delivery of ocular drugs, see Shirasaki, Y. “Molecular Design for Enhancement of Ocular Penetration” J. Pharm. Sci., Oct. 7, 2007 (Epub ahead of print); 1-35.

Corneal absorption represents a more efficient way to deliver intraocular drugs, but this route is rate limited by the cornea epithelium. Thus, there is a need to enhance trans-epithelial penetration, particularly of larger hydrophilic molecules, to provide for efficient intraocular delivery of drugs and other agents to the corneal stroma.

Accordingly, the instant disclosure provides methods of treating the cornea with agents that disrupt epithelial cell junctures. That disruption allows trans-epithelial diffusion of molecules that would otherwise enter the cornea only inefficiently. In addition, some of these same agents, which freely cross the epithelium due to their small molecular size (<500 daltons), penetrate the corneal stroma and react with deprotonated amine groups on the collagen fibrillar network resulting in dissociation of ionically bound proteoglycan bridges between collagen fibers. This temporarily destabilizes the fibrillar network and primes the fibrillar network for restabilization in the new, desired configuration. Stabilization molecules, such as decorin, may be applied to the corneal surface. The molecules penetrate the epithelium and bind to adjacent collagen fibers in the stroma, fixing the cornea in its new configuration to treat myopia, hyperopia and/or astigmatism.

It is known that various chemical agents will react with proteins to alter their chemical and physical characteristics. Generally, these chemical agents are used to modify proteins in solution. Several reviews discussing chemical modification are available, including Chemical Reagents for Protein Modification, Ed. R L Lunblad, CRC Press, Boca Raton, 1991 and G R Stark, Recent Developments In Chemical Modification And Sequential Degradation Of Proteins, Advances in Protein Chemistry, 24: 261-308, 1970. Specific chemical agents react with deprotonated free amines on proteins to replace the positive (NH₃⁺) charge with a chemical moiety exhibiting a negative charge or neutral charge. Other chemical agents react with deprotonated amines on proteins to replace a single positive charge with two positive charges. This change in net charge and charge density alters both the chemical and physical characteristics of the protein. This technology has been described in a pending patent publication (DeVore, et. al., U.S. Publication 20050106270).

For purposes of the present disclosure, any one of a wide variety of agents can be utilized to disrupt epithelial cell junctures and dissociate proteoglycans bridges between stromal collagen fibers. Chemical agents known to destabilize intact connective tissues have been described in U.S. patents issued to DeVore, et. al.

U.S. Pat. No. 4,969,912 (Kelman & DeVore) describes methods of solubilizing or partially solubilizing collagenous tissue, using acylation agents to form medical implants. U.S. Pat. No. 6,743,435 (DeVore & Ciaramentaro) describes methods of dispersing intact animal tissues using acylation agents.

U.S. Pat. No. 6,161,544 (DeVore and Oefinger) describes methods for destabilizing corneal tissues using acylation agents, such as glutaric anhydride. Patent application 20050106270 (DeVore and DeVore) describes methods for altering the chemical and physical characteristics of intact tissues using acylation agents. While these patents describe the use of chemical acylation agents to solubilize, disperse, and alter intact tissue, they do not describe the use of such agents to dissociate corneal epithelial junctures and/or to dissociate proteoglycans bridges between adjacent collagen fibers in corneal stroma.

### SUMMARY OF THE INVENTION

The disclosure describes methods of treating the cornea with agents that disrupt epithelial cell junctures. These methods can be used to enhance the ocular delivery of any molecule of interest, such as the ocular drugs used in the treatment of glaucoma and the stabilizing agents used in corneal reshaping. The disclosure also describes methods of treating the cornea with agents that dissociate bridging molecules from the collagen fiber units in the corneal stroma. This method facilitates stabilization of reshaped corneas curvature, such as results from orthokeratology.

The agents used in the methods of disrupting the epithelial cell junctures and in the methods of dissociating bridging molecules freely cross the epithelium due to their small molecular size (<500 daltons). Reactivity of the agents with deprotonated amines destabilizes the collagen fiber network, thereby priming the network for restabilization using exogenously applied stabilization molecules. Disruption of epithelial cell junctures allows the relatively large stabilization molecules to efficiently penetrate the epithelium and associate with adjacent collagen fibers in the stroma, fixing the cornea in a defined configuration. Stabilization of a reshaped corneal curvature resulting from procedures such as orthokeratology will provide a long-term, non-invasive treatment for conditions such as myopia, hyperopia, and astigmatism.

Accordingly, it is an object of the instant disclosure to provide methods for treating cornea that result in the disruption of epithelial cell junctures to facilitate diffusion of molecules into the corneal stroma. These methods are suitable for facilitating the entry of any molecule of interest into the cornea. In some embodiments, those molecules also dissociate the ionically bound bridging molecules from stromal collagen fibers to temporarily destabilize the stromal collagen network such that the network can be restabilized in a desired configuration. The methods can comprise administering a therapeutically effective amount of a single agent in a physiologically acceptable solution to both disrupt the epithelial cell junctures and dissociate the ionic bond bridging the stromal collagen fibers. In other embodiments, however, the methods comprise administering a therapeutically effective amount of a first agent in a physiologically acceptable solution to disrupt epithelial cell junctures to facilitate diffusion of molecules into the corneal stroma and then administering a second agent in a physiologically acceptable solution to dissociate ionically bound bridging molecules from stromal collagen fibers to temporarily destabilize such stromal collagen network such that the network can be restabilized in the desired configuration. In any of the disclosed methods, the cornea can also be primed for administering an agent capable of ionically bridging adjacent collagen fibers in the stroma to stabilize the cornea in its reshaped configuration. The disclosed methods can be used, either alone, in combination, or in combination with other methods, to treat myopia, hyperopia or astigmatism.

In one embodiment the disclosure provides methods of stabilizing the shape of a cornea, wherein the method comprises applying to the cornea an agent that disrupts the corneal epithelial junctures (“disrupting agent”) and applying to the cornea an agent that dissociates the molecular bridges between stromal collagen fibers (“dissociating agent”); then applying an agent that restabilizes the stromal collagen network to thereby stabilize the shape of the cornea. In some embodiments, the cornea has been reshaped using an orthokeratology procedure.

In other embodiments, the disclosure provides methods of enhancing ocular drug delivery, comprising applying to the cornea an agent that disrupts the corneal epithelial junctures before applying the ocular drug. The ocular drug, the agent that disrupts the corneal epithelial junctures, or both the ocular drug and the agent that disrupts the corneal epithelial junctures can be applied using an applicator applied to the surface of the cornea. Consistent with the other methods, this method may further comprising applying to the cornea an agent that dissociates the molecular bridges between stromal collagen fibers before applying the ocular drug. The ocular drug can be any ocular drug, but is often a hydrophilic ocular drug.

In each of the various methods, the disrupting agent may be an anhydride and the dissociating agent may be an anhydride, an acid chloride, a sulfonyl chloride, or a sulfonic acid. In certain embodiments, the disrupting agent is chosen from maleic anhydride, succinic anhydride, glutaric anhydride, citractonic anhydride, methyl succinic anhydride, itaconic anhydride, methyl glutaric anhydride, dimethyl glutaric anhydride, or phthalic anhydride. Similarly, in certain embodiments, the dissociating agent is chosen from acetic anhydride, butyric anhydride, or propionic anhydride. In those methods comprising application of a stabilizing agent, the stabilizing agent may be decorin, biglycan, keratocan, lumican, mimican, fibromodulin, Type VI collagen, Type X collagen, Type XII collagen, or Type XIV collagen, depending upon the particular embodiment. In certain embodiments, the stabilizing agent is human recombinant decorin.

Other objects, features and advantages of the invention shall become apparent as the description thereof proceeds when considered in connection with the accompanying illustrative drawings.

### BRIEF DESCRIPTION OF THE DRAWINGS

FIG. 1 shows the penetration of fluorescent-labeled decorin into cornea following controlled application of decorin directly to the central cornea. FIG. 1A shows a cornea pretreated with glutaric anhydride. FIG. 1B shows a control cornea.

FIG. 2 shows the penetration of fluorescent-labeled decorin into cornea following controlled application of decorin directly to the central cornea. FIG. 2A shows a cornea pretreated with acetic anhydride. FIG. 2B shows a control cornea.

FIG. 3 shows transmission electron micrographs of corneas. In FIG. 3A, the cornea was supplemented with decorin treatment. FIG. 3B shows a control cornea.

FIG. 4 shows transmission electron micrographs of corneas. FIG. 4A shows proteoglycan links between collagen fibers. These links are absent in corneas treated by acylation as shown in FIGS. 4B and 4C.

### DETAILED DESCRIPTION

The present disclosure provides methods of increasing the permeability of corneal epithelium to facilitate the diffusion of molecules, such as collagen binding molecules or various ocular drugs, into the corneal stroma. It should be noted that although “drug” can have special meaning in other contexts, as used herein it is a general term used to encompass any agent, whether chemical or biologic, that it is intentionally applied to the eye.

As already noted and reviewed in Shirasaki, Y. “Molecular Design for Enhancement of Ocular Penetration” J. Pharm. Sci., Oct. 7, 2007 (Epub ahead of print); 1-35, there are significant barriers that limit drug absorption to the anterior segment of the eye. In addition, topically applied drugs are diluted by tear liquid and rapidly removed by tear turnover and blinking. In general, therefore, only 1-7% of the dose of a drug enters the aqueous humor after topical administration. Drug in tear film is absorbed by corneal and noncorneal routes. Corneal penetration can occur via transcellular absorption or via paracellular absorption, but most drugs penetrate to the cornea via transcellular absorption. In this pathway, the drug is taken up by epithelial cells and transported across the cellular cytoplasm. Paracellular absorption, in contrast, involves transport through the junctures that occur between individual cells. But because the corneal epithelium exhibits such tight junctures, paracellular permeability is limited. Drugs therefore pass through the conjunctiva and sclera.

Most small molecular weight, lipophilic drugs are absorbed via the corneal route. However, the cornea is very tight tissue and the corneal epithelium is a lipophilic membrane, with tight junctures that act as a barrier to drug absorption. Thus, even though a lipophilic drug may pass through the lipophilic corneal epithelium, its penetration through the stroma will be limited because the stroma is hydrophilic. Hydrophobic drugs suffer from difficulty in penetrating the lipophilic corneal epithelium.

Thus there is a need in the art to provide more efficient delivery of ocular drugs. The disclosure provides methods that increase epithelial permeability. This enhances the delivery of compounds, including hydrophilic compounds and compounds of high molecular weight, to the aqueous humor. Certain hydrophilic drugs having low epithelial permeability will have dramatically improved efficacy using this technique.

Although the method can be applied to enhance delivery of any ocular drug, mention may be made of several classes of compounds that are often used as ocular drugs. One such class of ocular drugs is the antiviral agents. Among antiviral agents, drugs such as acyclovir and ganciclovir, which have low ocular permeability due to their hydrophilic nature may particularly benefit from the disclosed method of enhancing the delivery of ocular drugs. Another class of ocular drugs is the anti-inflammatory agents. Examples include the non-steroidal anti-inflammatory drugs (NSAIDs), such as diclofenac, bromfenac, flurbiprofen, pranoprofen, nepafenac, and ketorolac tromethamine and the steroids, such as prednisolone and dexamethasone. Anti-glaucoma agents are yet another class of ocular drugs. These include: carbonic anhydrase inhibitors, such as acetazolamide, methazolamide, and ethoxzolamide; certain beta-blockers with low corneal permeability coefficients, such as Acebutolol, Nadolol, Atenolol, and Sotalol; α₂-agonists such as apraclonidine; and postaglandin F_{2α} derivatives modified to decrease ionization in tear fluid. Anti-infective agents are yet another class of ocular drugs that includes examples with low corneal permeability. Some non-limiting examples of anti-infectives are the fluoroquinolones, such as ciprofloxacin. Allergy drugs are yet another class of ocular drugs. Other examples of ocular drugs can be found in Shirasaki, Y. “Molecular Design for Enhancement of Ocular Penetration” J. Pharm. Sci., Oct. 7, 2007 (Epub ahead of print); 1-35.

The disclosure also provides methods of temporarily destabilizing the collagen fibrillar network of the stroma. These methods are often used in combination with the methods of disrupting the epithelial cell junctures, although the disclosure also contemplates that they may be used on their own. Temporary destabilization of the stroma facilitates its restabilization in a new configuration, such as a configuration that improves visual acuity. Thus, among other methods, the disclosure provides methods of (1) disrupting corneal epithelial cell junctures to facilitate trans-epithelial diffusion and stromal penetration of agents, including high molecular weight agents that can stabilize collagen fibers and (2) dissociating molecular bridges between stromal collagen fibers to prime the collagen network for restabilization by exogenously applied stabilization molecules.

In many embodiments, the methods comprise administering therapeutically effective amounts of acylation or acetylation reagents to the surface of the cornea of the eye. As discussed in more detail below, acylation or acetylation agents include anhydrides, acid chlorides, sulfonyl chlorides, and sulfonic acids.

Usually, the agents are administered to the surface of the cornea after the cornea has been treated with a solution to deprotonate free amines on corneal proteins. The deprotonation solutions exhibit a pH range of from 7.5-10.0, often from 8.0-9.0, and usually from 8.3-8.7. They generally include buffer solutions and salt solutions exhibiting a pH in the desired range, such as buffers that are mixtures of dibasic sodium phosphate and monobasic sodium phosphate, or disodium phosphate alone. The concentration of the buffers and solutions ranges from 0.05-1.0M, is often between 0.1-0.7M, and is usually between 0.2 and 0.5M.

The various solutions used in the practice of the methods can be administered to the eye without any attempt to limit which tissues of the eye they contact. However, the deprotonation solutions and the solutions containing the disrupting and/or dissociation agents will often be administer using a device placed on the corneal surface to limit corneal surface exposure. Non-limiting examples of applicators for use in applying solutions to the corneal surface are described in the co-pending provisional application entitled “APPARATUS TO IMPROVE LOCALIZED CONCENTRATION OF FLUIDS IN OCULAR ENVIRONMENTS” to Bruce DeWoolfson and Michael Luttrell, provisional application No. 61/064,731, filed Mar. 24, 2008, incorporated herein by reference in its entirety.

When an acetylation or acetylation agent is used to disrupt the epithelial cell junctures, it can be an acylation or acetylation agent that replaces a negative charge with a negative charge, that replaces a positive charge with a neutral charge, or that replaces a positive charge with two positive charges.

When an acetylation or acetylation agent is used to dissociate molecular bridges between stromal collagen fibers, it can be any acylation or acetylation agent that replaces a negative charge with a neutral charge. This helps prevent increased protein hydration and subsequent corneal swelling.

One area in which it is important to stabilize the shape of the cornea is following an orthokeratology procedure. Accordingly, the disclosure also provides a method of stabilizing human cornea following an orthokeratology procedure. These methods comprise disrupting corneal epithelial cell junctures to facilitate trans-epithelial diffusion and stromal penetration of high molecular weight agents to stabilize collagen fibers and dissociating molecular bridges between stromal collagen fibers to prime the collagen network for restabilization by exogenously applied stabilization molecules. In some embodiments, the methods comprise administering therapeutically effective amounts of acylation or acetylation reagents to the surface of the cornea of the eye. After disruption and dissociation, a stabilization agent is then applied to the cornea to penetrate the stroma and restabilize the stromal collagen network in the configuration created by orthokeratology. Ideally, this procedure produces emmetropia.

The stabilizing agent used in the disclosed methods can be any molecule that can be applied exogenously to stabilize the stromal collagen network. However, the stabilizing agent is often a small leucine-rich proteoglycan (SLRP), which includes decorin, biglycan, keratocan, lumican, mimican, and fibromodulin, or a fibril associated collagen molecule with interrupted triple helices (FACIT), which includes Type VI, Type X, Type XII, and Type XIV collagen. Often, the stabilization molecule is human recombinant decorin. The decorin is generally applied in a solution in which the concentration of the human recombinant decorin solution is from about 0.05 to about 25 mg/mL. Often, the concentration is from about 1 to about 10 mg/mL, and usually it is from about 2 to about 6 mg/mL. The volume used for applying the stabilizing agent, such as the human recombinant decorin, generally ranges from about 0.05 to 5 mL. Often it is from about 0.1 to about 2.0 mL, and in many cases the volume is from about 0.2 to about 1.0 mL.

### Chemical Agents to Disrupt Epithelial Cell Junctures and to Dissociate Molecular Bridges between Collagen Fibers

For purposes of the present disclosure, there is a wide variety of agents that can be utilized to disrupt epithelial cell junctures and/or to dissociate molecular bridges between stromal collagen fibers. In the disclosed methods, an agent that disrupts the corneal epithelial junctures may be referred to as a “disrupting agent”. Similarly, an agent that dissociates the molecular bridges between stromal collagen fibers may be referred to as a “dissociating agent”.

Chemicals and pharmaceuticals that have been reported to disrupt epithelial cell junctures include EDTA, periodates, high concentration urea compounds, magnesium chloride, and organic solvents. However, there are limited number of agents that have been reported to be capable of dispersing intact tissue or dissociating molecular bonding molecules from other tissue components (e.g., acylating agents, see DeVore, et. al. patents). For example, U.S. Pat. Nos. 3,760,807, 3,776,230 and 3,831,604 to Neefe collectively describe the use of chemicals such as proparacaine hydrochloride, dyclonine hydrochloride, chlorine in solution, and the use of proteolytic enzymes all to soften the collagenous tissues of the cornea. Also, U.S. patents to Harris describe the use of enzymes, such as hyaluronidase, for softening of corneal collagen tissues. Even further still, U.S. Pat. Nos. 4,713,446, 4,851,513, 4,969,912, 5,201,764, 5,354,336 and 5,492,135 to Kelman and DeVore and DeVore patent application publication 20050106270 each describe various chemical agents for treating and/or softening both natural and artificial collagen-based materials for ophthalmic uses. The teachings of all of these patents with respect to chemical destabilizing agents are incorporated herein by reference. While incorporated herein, the teachings of these patents are not intended to limit the scope of the term destabilizing reagent, and the listings recited therein are not intended to be limiting.

Thus, there are several chemical and pharmaceutical agents that have been reported to disrupt epithelial cell junctures. However, there are only a limited number of agents that have been reported to disperse intact tissue (e.g., acylation agents, see DeVore et. al. patents). The families of reagents that produce these actions include anhydrides, acid chlorides, sulfonyl chlorides, and sulfonic acids. But, no agents have been reported that both disrupt epithelial cell junctures and dissociate the molecular bridges (e.g., FACITS and SLRPS) that exist between stromal collagen fibers. Accordingly, the agent used to disrupt the epithelial cell junctures is often not the same as the agent used to dissociate FACITS and SLRPS from stromal collagen fibers. However, the disclosure also expressly contemplates using the same agent to accomplish both functions.

The following lists of agents are intended to be representative of types of agents that disrupt epithelial cell junctures and/or dissociate FACITS and SLRPS from stromal collagen fibers. These lists are exemplary only, and are not intended to be limiting.

For epithelial cell disruption, suitable anhydrides include agents that change the net charge from positive to negative. These agents include, but are not limited to, anhydrides including maleic anhydride, succinic anhydride, glutaric anhydride, citractonic anhydride, methyl succinic anhydride, itaconic anhydride, methyl glutaric anhydride, dimethyl glutaric anhydride, phthalic anhydride, and many other such anhydrides. Acid chlorides include, but are not limited to, oxalyl chloride, malonyl chloride, and many others. Sulfonyl chlorides include, but are not limited to, chlorosulfonylacetyl chloride, chlorosulfonylbenzoic acid, 4-chloro-3-(chlorosulfonyl)-5-nitroebnzoic acid, 3-(chlorosulfonyl)-P-anisic acid, and others. Sulfonic acids include, but are not limited to, 3-sulfobenzoic acid and others.

Other agents can change the net charge from one positive to two negatives per reacted site. Examples of such agents include, but are not limited to, 3,5-dicarboxy-benzenesulfonyl chloride and others.

Still other agents can be used to change the net charge from positive to neutral per reacted site. Examples of those agents include, but are not limited to, anhydrides including acetic anhydride, chloroacetic anhydride, propionic anhydride, butyric anhydride, isobutyric anhydride, isovaleric anhydride, hexanoic anhydride, and other anhydrides; acid chlorides including acetyl chloride, propionyl chloride, dichloropropionyl chloride, butyryl chloride, isobutyryl chloride, valeryl chloride, and others; sulfonyl chlorides including, but not limited to, ethane sulfonyl chloride, methane sulfonyl chloride, 1-butane sulfonyl chloride and others.

For dissociation of FACITS and/or SLRPS from stromal collagen fibers, the disclosure provides, among other agents, those agents that disperse tissue but do not increase tissue hydration (cause swelling) or increase the biomechanical strength.

Agents that dissociate FACITS and/or SLRPs from stromal collagen fibers include agents that change the net charge from positive to negative. These agents include, but are not limited to, anhydrides, acid chlorides, sulfonyl chlorides, and sulfonic acids. Examples of anhydrides include maleic anhydride, succinic anhydride, glutaric anhydride, citractonic anhydride, methyl succinic anhydride, itaconic anhydride, methyl glutaric anhydride, dimethyl glutaric anhydride, phthalic anhydride, and many other such anhydrides. Acid chlorides include, but are not limited to, oxalyl chloride, malonyl chloride, and many others. Sulfonyl chlorides include, but are not limited to, chlorosulfonylacetyl chloride, chlorosulfonylbenzoic acid, 4-chloro-3-(chlorosulfonyl)-5-nitrobenzoic acid, 3-(chlorosulfonyl)-P-anisic acid, and others. Sulfonic acid reagents include, but are not limited to, 3-sulfonylbenzenoic acid, and others.

Other agents can change the net charge from one positive to two negatives per reacted site. Specific agents include, but are not limited to, 3,5-dicarboxybenzenesulfonyl chloride, and others.

In certain embodiments, the disclosure provides methods in which a simple anhydride, such as glutaric anhydride, is used to disrupt epithelial cell junctures and a simple anhydride, such as acetic anhydride, butyric anhydride or propionic anhydride, is used to dissociate FACITS and SLRPS from stromal collagen fibers. Each of these anhydrides hydrolyzes into innocuous compounds, many common in intermediary metabolism.

For use in the disclosed methods, the agents are generally diluted in a physiologically acceptable solution at slightly alkaline pH, such as disodium phosphate solution at a pH of approximately 8.5, or in another buffer providing a pH between about 8.3 and about 8.8. The solutions are then applied directly to the corneal surface in an applicator placed on the corneal surface. Non-limiting examples of applicators for use in applying solutions to the corneal surface are described in the co-pending provisional application entitled “APPARATUS TO IMPROVE LOCALIZED CONCENTRATION OF FLUIDS IN OCULAR ENVIRONMENTS” to Bruce DeWoolfson and Michael Luttrell, provisional application No. 61/064,731, filed Mar. 24, 2008, incorporated herein by reference in its entirety. The agents should be applied to the tissue surface after first priming the tissue with the slightly alkaline pH solution or buffer. Acylation agents either react with proteins that have first been deprotonated or hydrolyze into acids.

Thus, although a variety of different agents could be utilized as cell juncture disrupting agents and stromal destabilizing agents, this disclosure focuses on certain families of such agents, including anhydrides, acid chlorides, sulfonyl chlorides, and sulfonic acids. As previously noted, when an acylation agent is used, the type of acylation agent that results in cell juncture disruption may be different from the type of acylation agent that results in the dissociation of molecular bridges between stromal collagen fibers without corneal swelling. The latter type of agent is limited to those that substitute a non-charged moiety or (a positively charged moiety) to a deprotonated amine. Substitution with a negatively charged moiety has been shown to result in “hardening” of the treated tissue.

The following lists of agents are intended to be representative of these types of agents for dissociating molecular bridges between stromal collagen fibers without causing corneal swelling. The list is exemplary only, and it is not intended to be limiting.

Suitable, but non-limiting examples of potential anhydrides include: Acetic Anhydride, Propionic Anhydride, Methacrylic Anhydride, Butyric Anhydride, Isobutryic Anhydride, Valeic Anhydride, Hexanoic Anhydride, Decanoic Anhydride, Dodecanoic Anhydride, Myristic Anhydride, Palmitic Anhydride, and Oleic Anhydride.

Suitable, but non-limiting examples of potential acid chlorides include: Propionyl Chloride, Methacryloyl Chloride, Acryloyl Chloride, Methacryloyl Chloride, Butyryl Chloride, Isobutyryl Chloride, Valeryl Chloride, Isovaleryl Chloride, Hexanoly Chloride, and Heptanoly Chloride.

Suitable, but non-limiting examples of potential sulfonyl chlorides include 1-Hexadecanesulfonyl Chloride, 4-(Hexadecyloxy)benzenesulfonyl Chloride, Pentamethylbenzenesulfonyl Chloride, 4-Tert-Butylbenzenesulfonyl Chloride, Tolulenesulfonyl Chloride, and 2,5 Dimethylbenzenesulfonyl Chloride.

Suitable, but non-limiting examples of potential sulfonyl acids include 5-Tridecyl-1-2, Oxathiolane-2,2-Dioxide. All of the chemicals listed above are available from Sigma-Aldrich Chemical Company (St. Louis, Mo.).

Of the above agents, the simple anhydrides, e.g., acetic anhydride, butyric anhydride or propionic anhydride, may be used in many embodiments to dissociate molecular bridging of stromal collagen fibers since each of these anhydrides hydrolyze into rather innocuous compounds.

Among those agents that disrupt epithelial cell junctures, many embodiments utilize the simple anhydrides, e.g., maleic anhydride, succinic anhydride, glutaric anhydride, citractonic anhydride, methyl succinic anhydride, itaconic anhydride, methyl glutaric anhydride, dimethyl glutaric anhydride, phthalic anhydride. However, many other such anhydrides could be used.

Although agents can be administered to the cornea by applying them in solution form to the eye, that route of absorption involves penetration across the sclera and conjunctiva into intraocular tissues. As discussed, this is an inefficient method of delivering agents to the cornea because when the agent penetrates the surface of the eye beyond the corneal-scleral limbus, it is picked up by local capillary beds and removed by the general circulation. Generally, less than 1% of ophthalmic solutions administered via the noncorneal route reach the aqueous humor.

Corneal absorption represents a more efficient way to deliver intraocular drugs, but this route is rate limited by the corneal epithelium. In general, molecules larger than about 500 daltons penetrate the epithelium only inefficiently, if at all. However, many ocular drugs and agents used in procedures to correct visual acuity are larger than 500 daltons in size. For a general review of ophthalmic delivery, see Ophthalmic Drug Delivery Systems, Ed: AK Mitra, Marcel Dekker, Inc., 1993.

The disclosed method of disrupting the epithelial cell junctures can be used to facilitate delivery of molecules larger than 500 daltons to the corneal stroma. For example, as discussed, the methods can be used to facilitate stromal delivery of human recombinant decorin, which is about 40,000 daltons. Even larger molecules can also be delivered using the disclosed methods.

The deliver efficiency of agents to the cornea can also be improved by administering the agent of interest to the cornea by direct administration of a solution containing it into an applicator applied to the surface of the cornea. This application technique exposes the central core of the cornea to the agent, but prevents exposure to the corneal periphery. The agents generally are dissolved or diluted in a physiologically acceptable solution immediately prior to treatment and placed into a syringe for injection into an applicator. Non-limiting examples of applicators for use in applying solutions to the corneal surface are described in the co-pending provisional application entitled “APPARATUS TO IMPROVE LOCALIZED CONCENTRATION OF FLUIDS IN OCULAR ENVIRONMENTS” to Bruce DeWoolfson and Michael Luttrell, provisional application No. 61/064,731, filed Mar. 24, 2008, incorporated herein by reference in its entirety. The solution is then injected into the applicator exposing the surface of the cornea for about 2 seconds to about 1 minute, often from about 15 seconds to about 45 seconds, and usually from about 25 seconds to about 35 seconds. Direct corneal deliver can be used to facilitate the delivery of any agent to the cornea.

As noted, the various methods described can be used alone with various benefits. Thus, for example, methods of disrupting epithelial cell junctures can be used to facilitate the stromal delivery of any ophthalmic drug or other molecule for which intrastromal delivery is desired. However, the methods can also be used in combination with each other, and even as part of a larger procedure. As an illustrative, non-limiting, example, the following sequence has been used to disrupt epithelial cell junctures, to dissociate bridging molecules from stromal collagen fibers and to restabilize corneal structure.
1. Apply drops of topical anesthetic for about 2 minutes;
2. Apply pretreatment buffer to the eye for about 30 seconds;
3. Apply epithelial cell juncture disrupting anhydride reagent in pretreatment buffer for about 30 seconds;
4. Apply a second application of pretreatment buffer for about 30 seconds;
5. Apply molecular bridge dissociating reagent in pretreatment buffer to the eye for about 30 seconds;
6. Rinse thoroughly with sterile buffer solution or sterile physiological saline solution.
7. Apply solution of restabilizing molecules for about 30 seconds
8. Rinse thoroughly with sterile buffer solution or sterile physiological saline solution
   This general method is applicable to any situation in which it would be desirable to stabilize the corneal shape. One non-limiting example of such an application is the stabilization of corneal tissue following orthokeratology.

Of course, variations on this methodology are possible, particularly with respect to application times, buffer systems, and the exact pH of the solutions. Examples of treatments to disrupt epithelial cell junctures to permit penetration by high molecular weight agents, such as stabilization molecules, are described below.

### Example 1

### Confocal Microscopy to Evaluate Decorin Penetration into Human Donor Corneal Tissue

Confocal Microscopy of glutaric anhydride treated cornea. Four donor corneas were obtained from Insight Biomed (Minneapolis, Minn.). All cornea tested negative for viral contamination. Corneas were rejected for transplantation due to storage expiration, low endothelial cell counts or other factors not related to epithelial cell integrity and stromal structure. All corneas were examined by slit-lamp microscopy for epithelial cell integrity. Corneas were stored in Optisol (Bausch & Lomb) for storage. Confocal microscopy was conducted at the Department of Surgical Research, Dartmouth-Hitchcock Medical Center, Lebanon, N.H. Two corneas were untreated controls and two were treated with glutaric anhydride followed by application of fluorescent-tagged human recombinant decorin. Human recombinant Decorin was prepared from CHO—S cells (Cardinal Health) and exhibited a concentration of 3.7 mg/mL in 10 mM NaPO₄ buffer+150 mM NaCl, pH 7.2. Fluorescent-tagged decorin was prepared by reacting decorin with Oregon Green 488 using a labeling kit from Molecular Probes.

All corneas were placed on a convex silicone pad and secured with pins. This allowed exposure to the corneal surface in a fixed position. All treatment solutions were administered using an applicator to localize exposure to the central corneal surface. Control cornea were treated with Proparacaine HCl for 1 minute followed by treatment with 0.5 mL of 0.2M sodium phosphate buffer (pH 8.3-8.5) for 30 seconds, 0.5 mL of saline rinse for 30 seconds, and then 0.1 mL of fluorescent-tagged human recombinant decorin. T reated cornea were treated with Proparacaine HCl for 1 minute, followed by treatment with 0.5 mL of 0.2M sodium phosphate buffer (pH 8.3-8.5) for 30 seconds, glutaric anhydride (dissolved immediately before application in sodium phosphate buffer (pH 8.3-8.5) at 5 mg/mL) for 30 seconds, saline rinsed, and then treated with 0.1 mL of fluorescent-tagged human recombinant decorin. Control and treated corneas were examined for decorin diffusion into the stroma using the Zeis confocal microscope (Model LSM 510 Meta; C apo 40×, NA=1.2, Thornwood, N.Y.).

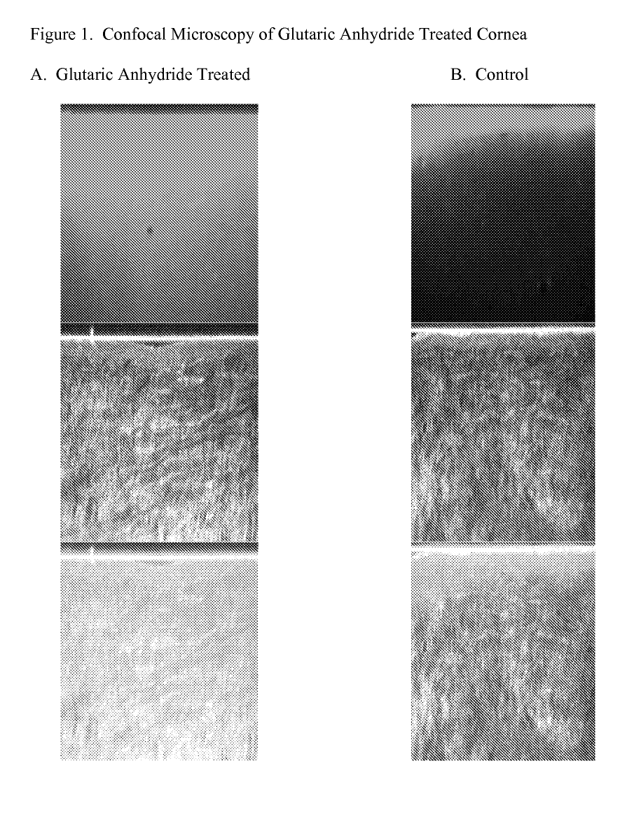

The results shown in FIG. 1 demonstrate the ability of glutaric anhydride to disrupt epithelial cell junctures so that human recombinant decorin (MW approximately 40,000 daltons) can diffuse into the cornea following direct application to the central cornea. As shown, decorin penetration into the control cornea was limited to the epithelium when it was applied directly to the central cornea and the epithelial cell junctures were not disrupted. Conversely, decorin penetrated the corneal stroma of the glutaric anhydride treated cornea. Thus, glutaric anhydride successfully disrupted the epithelial cell junctures, permitting diffusion of the 40,000 dalton decorin molecules.

Confocal Microscopy of acetic anhydride treated cornea. The protocol was the same as that described above for glutaric anhydride treatment. All treatment solutions were administered using an applicator to localize exposure to the central corneal surface. The applicator used to apply solutions to the corneal surface was similar in design to that described in the co-pending provisional application entitled “APPARATUS TO IMPROVE LOCALIZED CONCENTRATION OF FLUIDS IN OCULAR ENVIRONMENTS” to Bruce DeWoolfson and Michael Luttrell, provisional application No. 61/064,731, filed Mar. 24, 2008, incorporated herein by reference in its entirety. Control cornea were treated with Proparacaine HCl for 1 minute followed by treatment with 0.5 mL of 0.2M sodium phosphate buffer (pH 8.3-8.5) for 30 seconds, 0.5 mL of saline rinse for 30 seconds, and then 0.1 mL of fluorescent-tagged human recombinant decorin. Treated cornea were treated with Proparacaine HCl for 1 minute, followed by treatment with 0.5 mL of 0.3M sodium phosphate buffer (pH 8.3-8.5) for 30 seconds, acetic anhydride (3 μL) (diluted with 0.3M sodium phosphate buffer (pH 8.3-8.5) immediately before application) for 30 seconds, saline rinsed, and then treatment with 0.1 mL of fluorescent-tagged human recombinant decorin. Control and treated corneas were examined for decorin diffusion into the stroma using the Zeis confocal microscope (Model LSM 510 Meta; C apo 40×, NA=1.2, Thornwood, N.Y.).

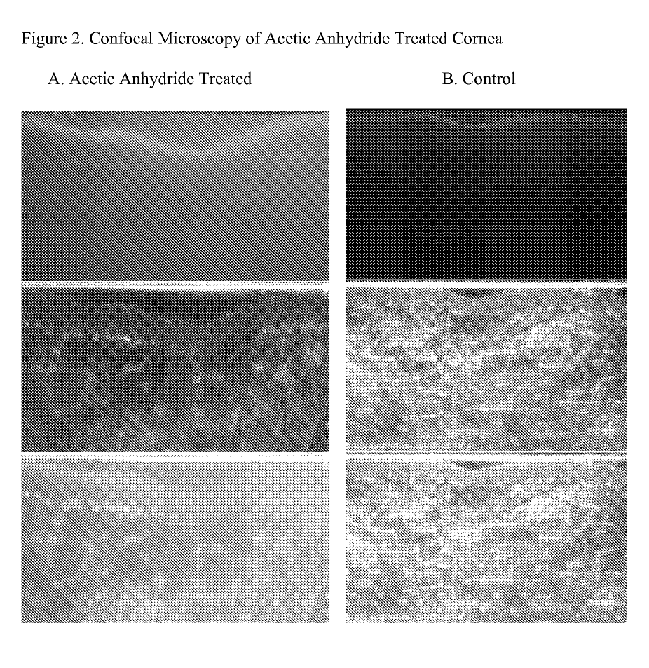

The results shown in FIG. 2 demonstrate the ability of acetic anhydride to disrupt epithelial cell junctures to permit diffusion of human recombinant decorin (MW approximately 40,000 daltons). As shown, decorin penetration into the control cornea was limited to the epithelium when the decorin was applied directly to the central cornea and the epithelial cell junctures were not disrupted. Conversely, decorin penetrated the corneal stroma of the acetic anhydride treated cornea. Therefore, acetic anhydride also successfully disrupted the epithelial cell junctures, permitting diffusion of the 40,000 dalton decorin molecules.

### Example 2

### Transmission Electron Microscopy to Evaluate Decorin Binding to Collagen Stromal Fibers in Human Donor Corneal Tissue

The following study was conducted at the Department of Surgical Research, Dartmouth-Hitchcock Medical Center, Lebanon, N.H. Five adult female cats were included in the study. All animals procured from Liberty Laboratories and were identified by ear tattoo. Ocular toxicity was determined by slit-lamp examination and measurement of endothelial cell structure. Decorin penetration into the corneal stroma was determined utilizing transmission electron microscopy. Cornea treated with decorin were reacted with Quinolinic Blue Stain (Cupromeronic blue) in buffered formalin. This reagent stains small proteoglycan structures such as decorin.

Cats were placed in three treatment groups. One eye from each group was treated with decorin. Three eyes were controls. Treated eyes were exposed to 50 μg of decorin for 1 day, 3 days or 5 days. Decorin solution was administered to the corneal using a sterile transfer pipet. An applicator was not used to localize the solution to the central corneal surface. All eyes were clinically evaluated immediately post-treatment and at days 2, 3, 5, and 8. One month after final treatment, eyes were reexamined and then enucleated. Each eye was sectioned. One half was placed in Formalin for subsequent histological analysis. The other half was again divided in half, one half prepared for Transmission Electron Microscopy.

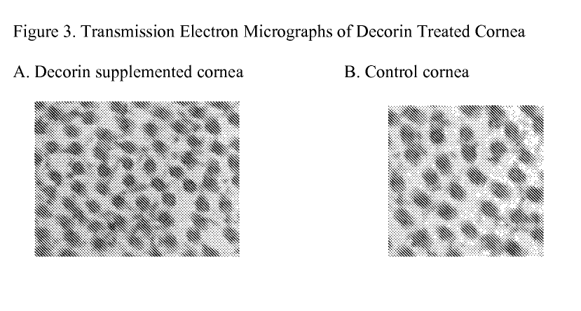

Transmission electron microscopy was conducted at the Department of Pathology, Robert Wood Johnson Medical Center, Rutgers University (New Brunswick, N.J.). FIG. 3 presents the micrographs for the decorin-supplemented (FIG. 3A) and the control (FIG. 3B) corneas. Note the increase in molecular bridges (links between fibers) between collagen fibers in the decorin supplemented cornea.

The following second study was conducted at the Department of Surgical Research, Dartmouth-Hitchcock Medical Center, Lebanon, N.H. Three adult female cats were included in the study. All animals procured from Liberty Laboratories and were identified by ear tattoo. Ocular toxicity was determined by slit-lamp examination and measurement of endothelial cell structure. Decorin penetration into the corneal stroma was determined utilizing transmission electron microscopy. Cornea treated with decorin were reacted with Quinolinic Blue Stain (Cupromeronic blue) in buffered formalin. This reagent stains small proteoglycan structures such as decorin.

Cats were placed in three treatment groups. One eye from two cats were untreated controls. All treatment solutions were administered using an applicator to localize exposure to the central corneal surface. The applicator used to apply solutions to the corneal surface was similar in design to that described in the co-pending provisional application entitled “APPARATUS TO IMPROVE LOCALIZED CONCENTRATION OF FLUIDS IN OCULAR ENVIRONMENTS” to Bruce DeWoolfson and Michael Luttrell, provisional application No. 61/064,731, filed Mar. 24, 2008, incorporated herein by reference in its entirety. Two eyes was treated with Proparacaine HCl, followed by treatment with 0.5M sodium phosphate buffer (pH 8.35), then with 3 mg of glutaric anhydride dissolved in 0.6 mL of sodium phosphate buffer, then with sodium phosphate buffer, followed by 1.5 μL of acetic anhydride diluted in 0.6 mL of sodium phosphate buffer, a second treatment with 1.5 μL of acetic anhydride in 0.6 mL of sodium phosphate buffer, a buffer rinse and finally treatment with 0.6 mL of human recombinant decorin (4.47 mg/mL).

Two eyes were treated as above but without a final treatment with decorin solution. All eyes were clinically evaluated immediately post-treatment. Three days following treatment, eyes were enucleated and the corneas removed, placed in formalin containing Cupromeronic blue.

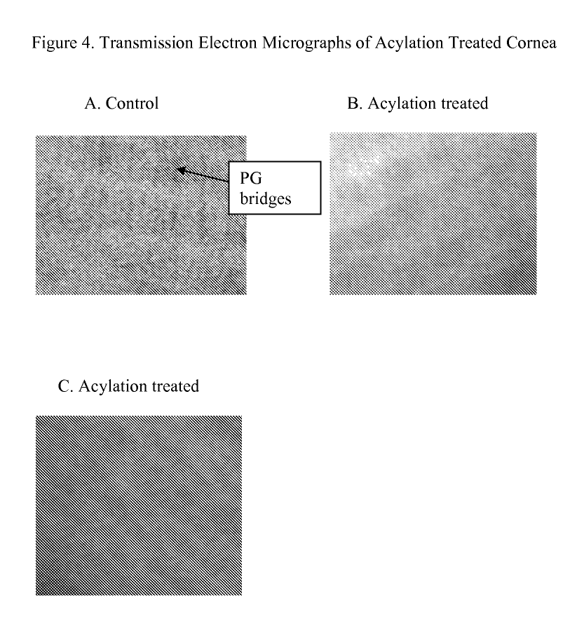

Transmission electron microscopy was conducted at the Department of Pathology, Robert Wood Johnson Medical Center, Rutgers University (New Brunswick, N.J.). FIG. 4 shows the micrographs of corneas following each treatment. FIG. 4A clearly shows the presence of proteoglycan links between adjacent collagen fibers. These links are absent in corneas treated with acylation agents, as shown in FIGS. 4B and 4C. Note the absence of bridging molecules in the acylation treated cornea.

### Example 3

### Effects of Acylation Treatments in Corneal Hysteresis in the Feline Model

Measurements of Corneal Hysteresis (CH) in the animals treated in Example 2. Corneal Hysteresis is a measure of the biomechanical strength of the cornea and is measured using the Reichert Ocular Response Analyzer. The Reichert Ocular Response Analyzer utilizes a dynamic bi-directional applanation process to measure the biomechanical properties of the cornea and the Intraocular Pressure of the eye. The basic output of the measurement process is a Goldmann-correlated pressure measurement (IOPG), and a new measure of corneal tissue properties called Corneal Hysteresis (CH). CH values are shown in Table 1.

TABLE 1
CH values for Feline Study #1
Corneal Hysteresis of Feline Cornea following
Acylation and Decorin Treatments

| | Animal IBT4 | | Animal QKU8 | | Animal QNO6 | |
|---|---|---|---|---|---|---|
| Treatment | Control | Treated | Treated | Treated | Control | Treated |
| Initial | ND | 4.0 | 6.4 | 7.0 | ND | 6.3 |
| Post- | ND | 7.0 | 8.0 | 7.5 | ND | 7.0 |
| Proparacaine | | | | | | |
| Post-GA | ND | 3.3 | 5.3 | 6.4 | ND | 6.4 |
| Post-AA #1 | ND | 3.2 | 7.0 | 5.7 | ND | 5.7 |
| Post-AA #2 | ND | 3.9 | 6.7 | 4.8 | ND | 3.8 |
| Post decorin | ND | 4.6 | 7.9 | | ND | |

As shown, acylation treatments reduced corneal hysteresis (CH) indicating “softening” of corneal structure due to dissociation of molecular links between collagen fibers. Subsequent application of decorin increased CH values to levels greater than initial values indicating “strengthening” of corneal structure.

Acylation treatments also disrupted corneal epithelial cell junctures permitting diffusion of human recombinant decorin into the stroma to restabilize the stromal collagen fiber network.

### Example 4

### Effects of Acylation Treatments in Corneal Hysteresis in the Feline Model

A second study was conducted to examine the effects of acylation on “softening” or destabilizing corneal structural integrity using the Reichert Ocular Response Analyzer. Five subject animals were included in this study. One eye of each animal was treated with the contralateral eye serving as the control. For three animal subjects treatment eyes were evaluated for corneal hysteresis using the Reichert Ocular Response Analyzer before treatment and then after treatment with Proparacaine HCL, acetic anhydride diluted in sodium phosphate solution at a pH of 8.3-8.5, and decorin. For two animal subjects, no acylation agent was applied to the cornea. Instead, a 4 mm trephine was used to penetrate the corneal epithelium to provide a track for decorin penetration. Corneal hysteresis results are shown below in Table 2.

TABLE 2
CH values for Feline Study #2

| | Animal | Animal | Animal | Animal | Animal |
|---|---|---|---|---|---|
| Treatment | AHH3 | QJD4 | RAF6 | BEA4 | IRH6 |
| Initial | 8.75 | 6.9 | 5.88 | 3.65 | 7.98 |
| Post AA | 5.73 | 3.9 | 4.15 | | |
| Post | 7.3 | 6.3 | 5.18 | 4.8 | 8.03 |
| decorin | | | | | |

As shown in Table 2, acylation treatments reduced CH values indicating corneal softening. Subsequent application of human recombinant decorin increased CH values indicating restabilization of corneal structure. Application of decorin in the trephined, but not acylation agent treated cornea, provided minimal increase in CH values. These results confirm the importance of dissociating molecular links between stromal collagen fibers prior to additional of stabilizing molecules, such as decorin.

### Exemplary Methodology

All treatment solutions are administered using an applicator to localize exposure to the central corneal surface. Non-limiting examples of applicators for use in applying solutions to the corneal surface are described in the co-pending provisional application entitled “APPARATUS TO IMPROVE LOCALIZED CONCENTRATION OF FLUIDS IN OCULAR ENVIRONMENTS” to Bruce DeWoolfson and Michael Luttrell, provisional application No. 61/064,731, filed Mar. 24, 2008, incorporated herein by reference in its entirety. Drops of Proparacaine HCl or a similar anesthetic are applied to the cornea for less than 5 minutes (e.g., for about 1-2 minutes). The corneal surface is then exposed to a small volume (e.g., about 0.1-1.0 mL) of a pretreatment buffer or solution at slightly alkaline pH ranging from 7.5-9.5. For example, the pH can range from between 8.0 and 9.0, or it can be between 8.2-8.7. Although the exact pH of the buffer may vary, the buffer must be sufficient to prevent the ultimate pH from dropping below 6.8. Suitable buffer solutions include sodium phosphate, and other buffer solutions providing a pH in the ranges disclosed above. Exposure time may range from 15 seconds to 2 minutes, although often the exposure time will be between 30 seconds and 1 minute. Following exposure to the pretreatment buffer or solution, the corneal surface is exposed to acylation agents. For example, the cornea is first exposed to glutaric anhydride (GA) or similar anhydrides, acid chlorides, sulfonyl chlorides, or sulfonic acids that are effective in disrupting epithelial cell junctures. Glutaric anhydride is a powder and must be rapidly dissolved in the pretreatment buffer before administration to the cornea. It is recommended to pulverize the glutaric anhydride powder using a mortar and pestle to reduce the particle size. This allows rapid dissolution. GA is dissolved at concentrations ranging from 1 mg/mL to 10 mg/mL. Often the concentration will be between 3 mg/mL and 5 mg/mL. The cornea is exposed to GA for, a period ranging from 15 seconds to 2 minutes. Often, the exposure time will be between 30 seconds to 1 minute. The corneal surface is then re-exposed to pretreatment buffer or solution for another short period of time, e.g., 30 seconds to 1 minute. At this point, the second acylation reagent is applied to the cornea surface to dissociate bridges or links between stromal collagen fibers. Again, treatment solutions are administered using an applicator to localize exposure to the central corneal surface. In many cases, the acylation agent is acetic anhydride (AA) or other anhydrides, acid chlorides, sulfonyl chlorides or sulfonic acids that do not result in corneal swelling. Acylation agents that impart a neutral charge to deprotonated amines are preferred for this treatment. Liquid acylation agents, such as acetic anhydride are diluted immediately before administration in pretreatment buffer or solution. The concentration depends on the particular acylation agent. When the acylation agent is AA, the concentration is generally between 1 and 5 μL per 0.6 mL of pretreatment buffer or solution. Usually, the concentration is 1-3 μL per 0.6 mL of pretreatment buffer or solution. The cornea is exposed to AA for less than 2 minutes, usually between 15 seconds to 1 minute, and in many cases the AA exposure time is between 20 seconds and 45 seconds. If desired, the AA treatment can be applied a second time. All treatment solutions are administered using an applicator to localize exposure to the central corneal surface. Non-limiting examples of applicators for use in applying solutions to the corneal surface are described in the co-pending provisional application entitled “APPARATUS TO IMPROVE LOCALIZED CONCENTRATION OF FLUIDS IN OCULAR ENVIRONMENTS” to Bruce DeWoolfson and Michael Luttrell, provisional application No. 61/064,731, filed Mar. 24, 2008, incorporated herein by reference in its entirety. Following AA treatment(s), the cornea is thoroughly rinsed with sterile saline, balanced salt solution, or other sterile physiological solutions. Finally, the corneal surface is exposed to a stabilizing (or restabilizing) agent. In many cases, the stabilizing agent is human recombinant decorin. Human recombinant decorin is usually applied at a concentration ranging from 1 to 5 mg/mL, often at a concentration of between 2 and 4 mg/mL. The cornea is exposed to decorin solution for usually less than 3 minutes, often from 15 seconds to 2 minutes, and in many cases from between 30 seconds and 1 minute. The eye is then flushed with sterile saline, balanced salt solution, or other sterile physiological solutions. These procedures are used to stabilize vision correction following orthokeratology procedures to provide a long-term, non-invasive treatment for myopia, hyperopia and astigmatism.

It can therefore be seen that the instant disclosure provides unique and effective methods for disrupting epithelial cell junctures to facilitate diffusion of hydrophilic and/or high molecular weight, molecules into the stromal matrix. It also provides unique and effective methods for dissociating molecular bridges or links between collagen fibers in the stromal matrix to permit restabilization of the matrix following corneal reshaping. The present methods of destabilizing the collagen fiber matrix of the lens will allow potential patients to have presbyopia treated in a matter of hours, without a significant recovery period. For these reasons, the instant disclosure is believed to represent a significant advancement in the art which has substantial commercial merit. The methods described have potential for use in conjunction with any technique that involves corneal reshaping, including methods to correct myopia, hyperopia and astigmatism.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A method of stabilizing the shape of a cornea, wherein the method comprises applying to the cornea an agent that disrupts the corneal epithelial junctures (“disrupting agent”) and applying to the cornea an agent that dissociates the molecular bridges between stromal collagen fibers (“dissociating agent”); then applying an agent that restabilizes the stromal collagen network to thereby stabilize the shape of the cornea.

2. The method of claim 1, wherein the cornea has been reshaped using an orthokeratology procedure.

3. The method of claim 1, wherein the disrupting agent is an anhydride and the dissociating agent is an anhydride, an acid chloride, a sulfonyl chloride, or a sulfonic acid.

4. The method of claim 3, wherein the disrupting agent is chosen from maleic anhydride, succinic anhydride, glutaric anhydride, citractonic anhydride, methyl succinic anhydride, itaconic anhydride, methyl glutaric anhydride, dimethyl glutaric anhydride, or phthalic anhydride.

5. The method of claim 3, wherein the dissociating agent is chosen from acetic anhydride, butyric anhydride, or propionic anhydride.

6. The method of claim 1, wherein the stabilizing agent is decorin, biglycan, keratocan, lumican, mimican, fibromodulin, Type VI collagen, Type X collagen, Type XII collagen, or Type XIV collagen.

7. The method of claim 6, wherein the stabilizing agent is human recombinant decorin.

8. A method of enhancing ocular drug delivery, comprising applying to the cornea an agent that disrupts the corneal epithelial junctures before applying the ocular drug.

9. The method of claim 8, further comprising applying to the cornea an agent that dissociates the molecular bridges between stromal collagen fibers before applying the ocular drug.

10. The method of claim 8, wherein the agent that disrupts the epithelium is chosen from maleic anhydride, succinic anhydride, glutaric anhydride, citractonic anhydride, methyl succinic anhydride, itaconic anhydride, methyl glutaric anhydride, dimethyl glutaric anhydride, or phthalic anhydride.

11. The method of claim 8, wherein the ocular drug is a hydrophilic drug.
